(19) United States
(12) Patent Application Publication
(10) Pub. No.: US 2022/0233441 A1
(43) Pub. Date: Jul. 28, 2022

(54) MICROEMULSION DELIVERY SYSTEMS FOR CANNABIS EXTRACTS AND TERPENES

(71) Applicant: Quicksilver Scientific, Inc., Louisville, CO (US)

(72) Inventors: Christopher W. Shade, Louisville, CO (US); Steven Tieu, Louisville, CO (US)

(73) Assignee: Quicksilver Scientific, Inc., Louisville, CO (US)

(21) Appl. No.: 17/672,988

(22) Filed: Feb. 16, 2022

Related U.S. Application Data
(63) Continuation of application No. PCT/US20/49168, filed on Sep. 3, 2020.
(60) Provisional application No. 62/896,820, filed on Sep. 6, 2019.

(51) Int. Cl. A61K 9/107; A61K 31/05; A61K 31/352; A61K 9/00; A61K 45/06; A61K 47/10; A61K 47/44

(52) U.S. Cl. CPC A61K 9/1075; A61K 31/05; A61K 31/352; A61K 9/0095; A61K 45/06; A61K 47/10; A61K 47/44; A61K 9/006

(57) ABSTRACT

Microemulsions are described where hydrophobic liquid droplets are distributed in a continuous hydrophilic liquid phase. The described microemulsions may be thought of as modified oil-in-water (MOIW) microemulsions, where both the “oil” and “water” phases of the microemulsion are modified. The oil phase droplets of the MOIW microemulsion are modified with alcohol and can solubilize oil-soluble species, including cannabis extracts and terpenes. The polar continuous “water” phase of the MOIW microemulsion is modified with a sugar or sugar alcohol.

### REFERENCE TO RELATED APPLICATIONS

This application is a continuation of International Application No. PCT/US20/49168, entitled “Microemulsion Delivery Systems for Cannabis Extracts and Terpenes”, filed Sep. 3, 2020, which claims the benefit of U.S. Provisional Application No. 62/896,820 entitled “Microemulsion Delivery Systems for Cannabis Extracts and Terpenes” filed Sep. 6, 2019, both of which are incorporated by reference in the entirety.

### BACKGROUND

Cannabinoids are compounds that act on the cannabinoid receptors in cells that alter neurotransmitter release. Cannabinoids include the endocannabinoids, which are produced naturally in the bodies of animals, phytocannabinoids, which are found in plants of the Cannabis genus and in some other plants, and synthetic cannabinoids that are synthesized. Type 1 cannabinoid receptors are found primarily in the brain and are absent from the part of the brain stem responsible for respiratory and cardiovascular function. Type 2 cannabinoid receptors are predominantly found in the immune system and appear to be responsible for the anti-inflammatory and possibly other therapeutic effects.

Phytocannabinoids are isolated from plants of the Cannabis genus, which is believed to include three species, Cannabis sativa, cannabis indica, and Cannabis ruderalis. Cannabis plants including less than 0.3% tetrahydrocannabinol (THC) by weight are commonly referred to as “hemp”, while plants including 0.3% or greater by weight THC are commonly referred to as marijuana. At least 113 different phytocannabinoids may be isolated from plants of the Cannabis genus. The phytocannabinoids are isolated in their “A” or acidic form and are then decarboxylated, often by heat, to their more biologically active, decarboxylated forms.

THC is the most famous cannabinoid as it binds to the Type 1 receptors and is considered psychoactive. Cannabidiol (CBD) is becoming a more commonly known non-psychoactive cannabinoid as it acts on the Type 1 and Type 2 receptors and is known to reduce pain and inflammation and calm some nerve responses, such as those associated with Dravet syndrome in children. Additionally, CBD may counteract cognitive impairment associated with THC use, including short term memory loss and may have additional anti-psychotic effects in addition to serving as an antioxidant. Cannabigerol (CBG) is another non-psychoactive cannabinoid that may have similar effects to CBD. Cannabichromene (CBC), Cannabinol (CBN), and Cannabitriol (CBT) are other cannabinoids that are being studied for potential biological activity.

While work remains ongoing regarding the health benefits of cannabinoids, pharmacological utility has been demonstrated or is likely to be demonstrated for the previously mentioned Dravet syndrome in children, Parkinson's disease, schizophrenia, anxiety disorder, and inhibiting the development of some cancer cells. As the human endocannabinoid system is involved in basic life functions, including appetite, immune response, reproduction, and pain management, the effects of cannabinoids on the human body are likely diverse. The ability of cannabinoids to prevent the overactivation of these functions may provide a reduction in progression or prevention of diseases based on overactivation of these functions.

Terpenes are extracted from plants, such as conifers, flowers, citrus fruits, and some insects, such as termites and swallowtail butterflies. From a molecular perspective, all terpenes include isoprene functionality and are a diverse class of organic molecules. In addition to historical use as fragrances, terpenes provide a basis for biologically active substances including Vitamin-A and steroids. Terpenes include compounds such as limonene, pinene, linalool, and beta-caryophyllene. Beta-caryophyllene, for example, has use as a fragrance and as an anti-inflammatory.

Oral delivery of cannabinoids and terpenes with conventional delivery systems can result in negligible blood concentration after 20-minutes of administration and not provide an effective bloodstream concentration, which is believed to be approximately 0.4 ng/mL and higher in the bloodstream for cannabinoids. In fact, for low absorbing individuals, effective bloodstream concentrations may not be reached after 90-minutes of administration, or at all, with conventional oral delivery systems without consuming undesirably large quantities of the conventional oral delivery system. Hence of the cannabinoids or terpenes consumed with conventional oral delivery systems, a significant majority of that consumed may be excreted and never used.

Emulsions are mixtures of two or more liquids that do not solubilize. Thus, the two or more liquids do not form a solution and an identifiable interface exists between the combined liquids. Emulsions may be macroemulsions, pseudo-emulsions, nanoemulsions or microemulsions. Emulsions may be used for parenteral delivery, ocular delivery, transdermal delivery, oral delivery, and the like.

FIG. 1A represents an example nanoemulsion droplet 100 having a single wall of phospholipids (monolayer) forming a hydrophilic exterior 120 and a hydrophobic interior 110. The monolayer wall of the nanoemulsion droplet 100 is formed from a single layer of phospholipids. The outer wall 120 is water soluble due to the phosphate functionality while the interior 110 is fat-soluble due to the alkyl functionality. FIG. 1B represents multiple of the nanoemulsion droplets 100 in a continuous phase 150.

FIG. 2A represents a microemulsion droplet 200 having a single wall of phospholipids (monolayer) forming a hydrophilic exterior 220 and a hydrophobic interior 210. As with the nanoemulsion droplets 100, the monolayer wall of the microemulsion droplet 200 is formed from a single layer of phospholipids. In relation to the represented nanoemulsion droplets 100, the microemulsion droplets 200 are substantially smaller in diameter—which is often the case for microemulsions. In fact, the diameter of the microemulsion droplets 200 are reduced to where non-polar tails 230 of the monolayer phospholipids are “crushed” into each other, thus forming a more “solid” interior hydrophobic barrier than in the case of the nanoemulsion droplets 100 as represented in FIG. 1. FIG. 2B represents multiple microemulsion droplets 200 in a continuous phase 250. Also represented in the continuous phase 250 are a few individual phospholipid molecules 260 not incorporated into the microemulsion droplets 200.

Transdermal creams are typically “pseudo-emulsions” with solid granules of the deliverable not fully solubilized in the droplets of the emulsion forming the cream. In contrast to the larger droplet macro- and pseudo-emulsions, the smaller droplets of nanoemulsions and microemulsions provide the potential to provide better delivery performance than conventionally available from macro- and pseudo-emulsions for either transdermal or oral adsorption.

While the high-energy mixing, in the form of pressure (including shear forces), temperature, and combinations thereof, used to form nanoemulsions can provide the smaller droplets of a microemulsion, such nanoemulsions are not thermally stable, thus are not shelf-stable microemulsions, and are like a macroemulsion in that the components of the nanoemulsion eventually separate into immiscible polar and non-polar liquids. Thus, as represented in FIG. 1 and FIG. 2, nanoemulsion droplets tend to be larger than microemulsion droplets as the nanoemulsion droplets continually expand in diameter after formation until the agglomerating droplets separate from the continuous phase.

Conventionally, macroemulsions, nanoemulsions, and microemulsions have been used for either oil-soluble or water-soluble deliverables. Cannabis extracts and terpenes are oil-soluble, but are absorbed relatively slowly and inconsistently through the gut when solubilized in oil. However, conventional oil-in-water (OIW) emulsions including cannabis extracts and some terpenes, regardless of emulsion form, generally form oil droplets that readily dissociate from the water phase of the emulsion with higher concentrations of cannabis extracts in the oil droplets. Such dissociation of the oil droplets from the water phase of conventional oil-in-water emulsions results in a significant loss in blood uptake rate and total blood delivery of the cannabinoids and terpenes when delivered by conventional oil-in-water emulsions. This is believed attributable to the fact that when the oil droplets of the emulsion dissociate from the water phase, the resulting dissociated emulsion becomes oil, water, and whatever remains of the emulsion, with the dissociated oil having the approximate delivery profile of oil alone, thus becoming slow and inconsistent. Thus, conventional oil-in-water emulsions including cannabis extracts and some terpenes tend to suffer from similar bloodstream uptake disadvantages to oil only formulations as the oil phase has dissociated from the water phase by the time the conventional emulsion is consumed.

As can be seen from the above description, there is an ongoing need for simple and efficient materials and methods for oral delivery systems for delivering cannabinoids and terpenes quickly and in higher concentration per consumed amount to the bloodstream. Conventional oil mixtures have traditionally been plagued with exceedingly slow, low, and inconsistent uptake attributed to the GI adsorption pathway. Conventional oil-in-water emulsion systems have traditionally had disadvantages including poor stability to cold and heat, particularly regarding maintaining the desired average droplet diameter in the emulsion, which is important for effective intra-oral delivery to the bloodstream, preventing phase separation of the oil and water components, and preventing dissociation of the deliverable and/or the oil from the emulsion. In addition to these disadvantages resulting in slow, poor, and inconsistent blood uptake of the deliverable, conventional emulsion systems also have the disadvantage of requiring too great a volume of the emulsion in relation to the mass or volume of the deliverable.

The microemulsions and methods of the present invention overcome at least one of the disadvantages associated with conventional oral delivery systems by allowing the convenient, rapid, efficient, and reproducible oral delivery of cannabinoids and terpenes to the bloodstream.

### SUMMARY

In one aspect, the invention provides a composition including an oil-soluble species; and a modified oil-in-water microemulsion including a modified oil phase and a modified polar continuous phase, where the oil-soluble species is solubilized in the modified oil phase, the modified oil phase comprising a phospholipid, a polyethylene glycol derivative, an oil, and an alcohol, and where the modified polar continuous phase includes a sugar or sugar alcohol and water.

In another aspect of the invention, there is a method of forming a modified oil-in-water microemulsion including an oil-soluble species, the method including combining a phospholipid, a polyethylene glycol derivative, an oil, and an alcohol to form an alcohol-lipid mixture; combining a sugar or sugar alcohol and water to form a modified polar continuous phase; and combining the oil-soluble species with the alcohol-lipid mixture and the modified polar continuous phase at atmospheric pressure to form the modified oil-in-water microemulsion.

In another aspect of the invention, there is a method of delivering an oil-soluble species to the bloodstream of a human subject, the method including introducing a modified oil-in-water microemulsion intra-orally to a human subject, and delivering the oil-soluble species to the bloodstream of the human subject, where within 20-minutes of the introducing the modified oil-in-water microemulsion to the human subject, approximately 1 mL the modified oil-in-water microemulsion provides the human subject a blood concentration from 0.3 to 1.5 ng/mL of the oil-soluble species.

Other systems, methods, features, and advantages of the invention will be, or will become, apparent to one with skill in the art upon examination of the following figures and detailed description. It is intended that all such additional systems, methods, features, and advantages be included within this description, be within the scope of the invention, and be protected by the claims that follow.

### BRIEF DESCRIPTION OF THE DRAWINGS

The invention can be better understood with reference to the following drawings and description. The components in the figures are not necessarily to scale and are not intended to accurately represent molecules or their interactions, emphasis instead being placed upon illustrating the principles of the invention.

FIG. 1A represents a nanoemulsion droplet having a single wall of phospholipids (monolayer) forming a hydrophilic exterior and a hydrophobic interior.

FIG. 1B represents multiple of the nanoemulsion droplets in a continuous phase.

FIG. 2A represents a microemulsion droplet having a single wall of phospholipids (monolayer) forming a hydrophilic exterior and a hydrophobic interior.

FIG. 2B represents multiple microemulsion droplets represented in a continuous phase.

FIG. 3 represents a method of making a MOIW microemulsion including an oil-soluble species.

FIG. 4 provides the results of a CBD blood uptake rate and concentration analysis in graphical form.

FIG. 5 provides the cumulative AUC values determined from the bloodstream concentration analysis in graphical form.

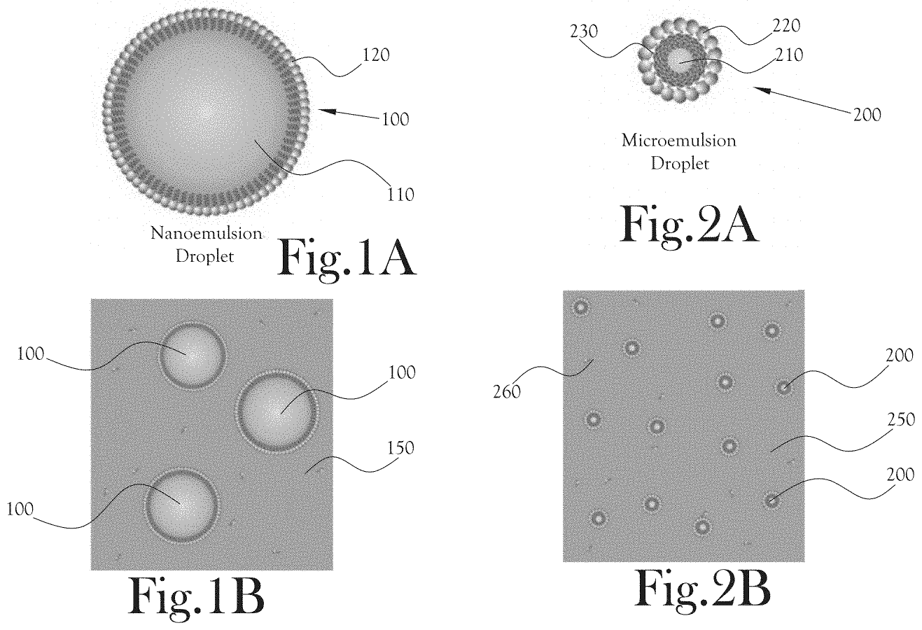

### DETAILED DESCRIPTION

Microemulsions are described where hydrophobic liquid droplets are distributed in a continuous hydrophilic liquid phase. In relation to conventional oil-in-water (OIW) microemulsions, the described microemulsions may be thought of as modified oil-in-water (MOIW) microemulsions, where both the “oil” and “water” phases of the microemulsion are modified. The oil phase droplets of the MOIW microemulsion are modified with alcohol and can better deliver oil-soluble species to the bloodstream than can oil blends or the oil phases of conventional oil-in-water (OIW) microemulsions. The polar continuous “water” phase of the MOIW microemulsion is modified with a sugar or sugar alcohol. Preferably, the modified polar continuous phase of the MOIW microemulsion is primarily a sugar or sugar alcohol phase. The modified oil phase droplets disperse into the modified polar continuous phase of the MOIW microemulsion.

The MOIW microemulsions can provide the uptake of the oil-soluble species to the bloodstream of a mammal through the oral and gastric mucosa, as well as transdermally through the skin. The MOIW microemulsion can orally deliver effective concentrations of the oil-soluble species to the bloodstream of an individual faster, such as within 20-minutes of introduction, even when the individual is low absorbing, than the oil phases of conventional OIW microemulsions. Also, of the oil-soluble species introduced, the MOIW microemulsion can deliver a significantly higher percentage of the oil-soluble species introduced orally to the bloodstream of the individual than the oil phases of conventional OIW microemulsions.

The modified polar continuous phase is believed to allow the modified oil phase droplets of the microemulsion to incorporate and retain a high alcohol content. Thus, the modified polar continuous phase is believed to force the alcohol into the oil and oil-soluble species residing within the interior of the monolayer walls formed from a phospholipid and a polyethylene glycol derivative, thus into the hydrophobic core of the modified oil droplets, while the modified polar continuous phase including the sugar or sugar alcohol and water resides external to the monolayer.

Unlike the water continuous phase of a conventional OIW emulsion, the sugar or sugar alcohol of the modified polar continuous phase does not readily form an azeotrope with alcohol, and thus has a reduced ability to extract the alcohol from the oil droplets in relation to water. The hydrophobic portion of the monolayer wall formed from the tails of the phospholipid and in combination with the polyethylene glycol derivative in the described ratios also are believed to reduce alcohol loss from the oil droplets in relation to conventional OIW emulsions.

The retained high alcohol content of the modified oil phase droplets provided by the combination of the modified polar continuous phase with the hydrophobic monolayer is believed to increase the solubility of the oil-soluble species in the modified oil droplets of the MOIW microemulsion in relation to conventional OIW emulsions. This enhanced solubility of the oil-soluble species in the modified oil droplets of the MOIW is believed to reduce dissociation (e.g. recrystallization, precipitation, and like—thus separation) of the oil-soluble species from the oil droplets of the MOIW microemulsion during storage thus making the MOIW microemulsion a shelf-stable microemulsion that preferably is visually clear. Additionally, the enhanced solubility of the oil-soluble species in the modified oil droplets of the MOIW is believed to deliver a greater amount of the oil-soluble species to the bloodstream per unit volume of the MOIW microemulsion in relation to conventional OIW emulsions.

In the MOIW microemulsion, modified oil phase droplets including the oil-soluble species have an average droplet diameter of 1 to 100 nanometers and a preferable average droplet diameter of 5 to 50 nanometers. More preferably, the modified oil phase droplets of the MOIW microemulsion have an average droplet diameter of 10 to 30 nanometers.

The oil-soluble species of the MOIW microemulsions may be delivered trans-mucosal (e.g. oral, intranasal, vaginal, or rectal) or transdermally via the MOIW microemulsion. Preferably, the MOIW microemulsion including the oil-soluble species is ingestible and edible.

The MOIW microemulsion preferably includes a ratio of phospholipid, to oil, to polyethylene glycol derivative, to alcohol, to sugar or sugar alcohol, and to water of 1:2:0.6-3.3:4:7-9:2-3.5 by weight, with deviations up to 20% by weight being included, and with deviations up to 10% by weight being more preferred, thus 1:2:0.6-3.3:4:7-9:2-3.5±20% by weight or 1:2:0.6-3.3:4:7-9:2-3.5±10% preferred by weight.

The oil-soluble species is preferably included in the MOIW microemulsion at a ratio of oil to oil-soluble species of 1:0.05 to 0.4 by weight, with a ratio of oil to oil-soluble species of 1:0.1 to 0.3 by weight being preferred with deviations up to 10% by weight being included, and with deviations up to 5% by weight being more preferred, thus 1:0.05 to 0.3±10% by weight or 1:0.05 to 0.3±5% preferred by weight.

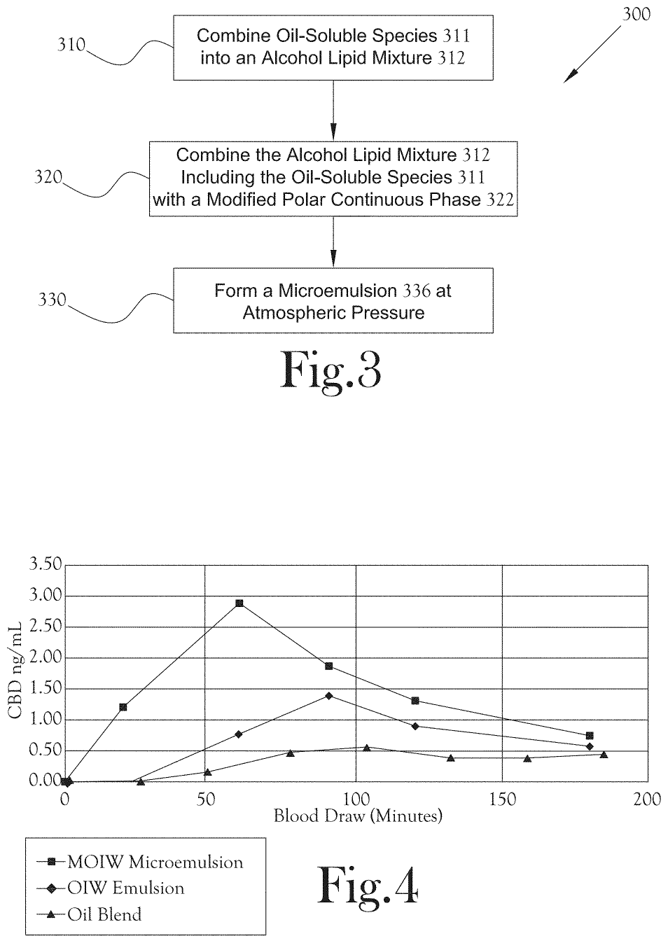

FIG. 3 represents a method 300 of making a MOIW microemulsion 336 including an oil-soluble species 311. In addition to the oil-soluble species 311, the microemulsion 336 may include additional deliverables that are soluble in water or oil.

In 310, the oil-soluble species 311 is combined into an alcohol-lipid mixture 312 including a polyethylene glycol derivative, a phospholipid, an oil, and an alcohol. In 320, the alcohol-lipid mixture 312 including the oil-soluble species 311 is combined with a modified polar continuous phase 322 including the sugar or sugar alcohol and water. The alcohol-lipid mixture 312 including the oil-soluble species 311 may be considered a modified oil phase dispersed in the modified polar continuous phase 322, which may be thought of as a modified water phase.

In 330, the microemulsion 336 including the oil-soluble species 311 is formed by mixing at atmospheric pressure. Unlike in nanoemulsions, the microemulsion 336 may be formed at atmospheric pressure without needing the energy of elevated pressures and/or shear forces to form. Although the microemulsion 336 could be formed using elevated pressure and/or shear forces as used in forming nanoemulsions, the result eventually will be the microemulsion 336, as unlike in a nanoemulsion that begins the dissociation process after formation—even if dissociation is very slow, the microemulsion 336 is thermally stable at room temperature and pressure after formation. Thus, formation of the microemulsion 336 dispenses with the undesirable use of elevated pressures and/or shear forces during formation, and is shelf-stable after formation.

While the method 300 represents the oil-soluble species 311 first being combined with the alcohol-lipid mixture 312, the alcohol-lipid mixture 312 and the polar continuous phase 322 may first be combined and the oil-soluble species 311 then added to form the microemulsion 336 (not shown). This step rearrangement is possible as the modified oil and modified polar continuous phases will “self-assemble” droplets including the oil-soluble species to form the microemulsion 336 at atmospheric pressure.

The oil-soluble species 311 is a liquid at room temperature and pressure, however at high purities, such as above 55% purity by weight, the oil-soluble species 311 may be or may include a crystalline solid. Once solubilized in oil, the oil-soluble species 311 will remain solubilized in the oil at room temperature and pressure. The oil-soluble species 311 preferably includes cannabis extracts and/or terpenes.

The oil-soluble species 311 is solubilized in the droplets of the microemulsion 336, thus in the alcohol lipid mixture 312. The alcohol-lipid mixture 312 is preferably configured so that the oil-soluble species 311 is more soluble in the alcohol-lipid mixture 312 than in the oil alone of the microemulsion 336.

Preferably, the oil-soluble species 311 constitutes from 1% to 6% of the microemulsion 336 by weight. However, to provide a visually clear emulsion with the widest range of oil-soluble species, weight percentages of the oil-soluble species 311 from 1% to 4% are preferred, with weight percentages from 1% to 3% being more preferred. When the oil-soluble species 311 is substantially terpenes, higher weight percentages within the 1% to 6% range may be used than when the oil-soluble species is substantially cannabis extract and maintain a visually clear emulsion. These stated weight percentages for the oil-soluble species 311 are in the context of the oil-soluble species 311 solubilized in the droplets of the microemulsion 336, not suspended in the emulsion liquid or otherwise dissociated from the droplets. An example of this situation is discussed in the context of Example 3 below, where a commercially purchased product either had an incorrect oil-soluble species content on the label, or approximately 70% of the oil-soluble species in the product had dissociated from the emulsion.

Cannabis extracts are oily extracts from a plant of the Cannabis genus. Preferable cannabis extracts include cannabidiol (CBD), tetrahydrocannabinol (THC), and other cannabinoids including cannabinol (CBN), cannabigerol (CBG), tetrahydrocannabivarin (THCV), cannabidivarin (CBDV), and cannabichromene (CBC). Preferred cannabis extracts include at least 30% by weight CBD and/or THC, while more preferred cannabis extracts include at least 60% by weight CBD and/or THC. Most preferred cannabis extracts include at least 80% by weight CBD and/or THC.

Preferable terpenes include monoterpenes (incorporate two isoprene units and have the molecular formula C₁₀H₁₆), monoterpenoids, diterpenes (incorporate four isoprene units and often have the molecular formula C₂₀H₃₂), and diterpenoids. Preferable terpenes for inclusion in the microemulsion 336 include limonene, pinene, linalool, beta-caryophyllene, retinol, phytol, myrcene, humulene, ocimene, terpinolene, geraniol, and geranylgeraniol.

The alcohol lipid mixture 312 optionally may include an alcohol-soluble deliverable that is a solid at room temperature and pressure. Thus, unlike the oil-soluble species 311 that is a liquid at room temperature and pressure or heated and solubilized in oil as previously described, the alcohol-soluble deliverable is a solid at room temperature and pressure. Preferably, the alcohol-soluble deliverable is less soluble in the oil than the oil-soluble species 311. Such alcohol-soluble deliverables are solubilized in the modified oil phase droplets of the microemulsion, thus in the alcohol lipid mixture 312 with the oil-soluble species 311.

Alcohol-soluble deliverables include some plant sterols, some polyphenols, and some anti-microbials. Preferable plant sterols include tribulus terrestris and yohimbe. Preferable polyphenols include resveratrol, pterostilbene, curcumin, Boswellia, and quercetin. Preferable anti-microbials include artemisinin, monolaurin, and Andrographis. Preferably, these alcohol-soluble deliverables are incorporated into the alcohol lipid mixture 312 of the microemulsion 336 as a solid in powder form.

The modified polar continuous phase 322 may include a water-soluble deliverable specie or species that is more soluble in water than the oil-soluble species 311. Such water-soluble deliverables are solubilized in the modified polar continuous phase 322 of the microemulsion 336. Thus, in the carrier liquid of the microemulsion 336.

The phospholipid and the polyethylene glycol derivative in combination form the boundary between the modified polar continuous phase and the interior of the modified oil phase droplets of the microemulsion 336. To maintain the desired alcohol concentration within the droplets, thus reducing the likelihood of losing the alcohol to the modified polar continuous phase and the associated dissociation of the oil-soluble species from the droplets, the phospholipid, polyethylene glycol derivative, and the ratio between the two are important, as previously discussed.

The phospholipid of the alcohol-lipid mixture 312 is a glycerophospholipid preferably isolated from lecithin. As the phospholipid is preferably a lecithin isolate, the named isolates preferably include 80% (w/w) of the specified phospholipid with the remaining constituents being one or more additional phospholipids isolated from the lecithin or other lecithin isolates. Preferred phospholipid lecithin isolates include phosphatidylcholine (PC), phosphatidylethanolamine (PE), phosphatidylinositol (PI), ceramide phosphoryl ethanolamine (Cer-PE), ceramide phosphoryl choline (SPH), and combinations thereof, with PC, PE, and combinations thereof being more preferred. However, all phospholipid lecithin isolates are unexpectedly not interchangeable in forming shelf-stable and visually clear MOIW microemulsions, as the phosphatidylserine (PS) and phosphatic acid (PA) isolates are not useful when both shelf-stable and visually clear MOIW microemulsions are desired. When the oil-soluble species 311 is cannabis extracts, the phospholipid is preferably PC.

The phospholipid may be present in the microemulsion 336 from 2% to 10% on a weight basis. Preferably, the phospholipid constitutes from 4% to 10% of the microemulsion 336 on a weight basis. When the oil-soluble species is cannabis extracts, the phospholipid constitutes from 4% to 8% of the microemulsion 336 on a weight basis.

The polyethylene glycol derivative of the alcohol-lipid mixture 312 may be a polyethylene glycol modified vitamin E, such as tocopheryl polyethylene glycol succinate 1000 (TPGS), polysorbate 40, polysorbate 60, or polysorbate 80. Preferably, the polyethylene glycol derivate is TPGS, polysorbate 60, or polysorbate 80. More preferably, the polyethylene glycol derivative is TPGS or polysorbate 80. When the oil-soluble species is cannabis extracts, the preferred polyethylene glycol derivative is TPGS.

The polyethylene glycol derivative may be present in the microemulsion 336 from 5% to 15% on a weight basis. Preferably, the polyethylene glycol derivative constitutes from 6% to 12% of the microemulsion 336 on a weight basis. When the oil-soluble species is cannabis extracts, the polyethylene glycol derivative constitutes from 9% to 11% of the microemulsion 336 on a weight basis.

TPGS, polysorbate 20, polysorbate 40, polysorbate 60, and polysorbate 80 are often thought of as interchangeable surfactants. This was determined not to be the case in the formation of the described microemulsion 336 when a shelf-stable and visually clear microemulsion is desired.

When used in conjunction with the phospholipid, TPGS resulted in shelf-stable and visually clear microemulsions at phospholipid to TPGS ratios of approximately 1:0.4 to 1:4 by weight, with preferred shelf-stable MOIW microemulsions being formed at ratios of 1:1.6 to 1:4 by weight. When used in conjunction with the phospholipid, polysorbate 20 did not form shelf-stable and visually clear microemulsions. When used in combination with the phospholipid, polysorbate 40 resulted in shelf-stable and visually clear microemulsions at PC to polysorbate 40 ratios of approximately 1:2 to 1:3 by weight, with preferred shelf-stable MOIW microemulsions being formed at a ratio of approximately 1:3 by weight. When used in combination with the phospholipid, polysorbate 60 resulted in shelf-stable and visually clear microemulsions at phospholipid to polysorbate 60 ratios of approximately 1:2 to 1:4 by weight, with preferred shelf-stable MOIW microemulsions being formed at a ratio of 1:2 to 1:3 by weight. When used in combination with the phospholipid, polysorbate 80 resulted in shelf-stable and visually clear microemulsions at phospholipid to polysorbate 80 ratios of approximately 1:0.4 to 1:4 by weight, with preferred shelf-stable MOIW microemulsions being formed at a ratio of 1:0.6 to 1:4 by weight.

These results establish that the multiple polyethylene glycol derivatives are unexpectedly not interchangeable in forming shelf-stable and visually clear MOIW microemulsions. In fact, polysorbate 20 is not useful. Furthermore, TPGS and polysorbate 80 are the preferred polyethylene glycol derivatives as in combination with the phospholipid, they provide the desired shelf-stable and visually clear microemulsions over the widest oil-soluble species concentration range.

The alcohol-lipid mixture 312 preferably includes at least one oil held within the phospholipid/polyethylene glycol derivative monolayer. The oil may be an MCT oil, a citrus oil, and combinations thereof. MCT oils are triglycerides whose fatty acids have an aliphatic tail of 6-12 carbon atoms. Preferable MCT oils include caproic acid (hexanoic acid), caprylic acid (octanoic acid), capric acid (decanoic acid), lauric acid (dodecanoic acid), and combinations thereof. More preferred MCT oils include caprylic acid, capric acid, and combinations thereof. Preferred citrus oils include orange oil, lemon oil, and combinations thereof. When the oil-soluble species is cannabis extracts, the oil is preferably a combination of caprylic and capric acids.

The oil may be present in the microemulsion 336 from 5% to 15% on a weight basis. Preferably, the oil constitutes from 7% to 13% of the microemulsion 336 on a weight basis. When the oil-soluble species is cannabis extracts, the oil constitutes from 9% to 11% of the microemulsion 336 on a weight basis.

The microemulsion 336 includes at least one alcohol. The preferable alcohol is food grade as the microemulsion 336 is preferably edible. Preferably, the alcohol is ethanol, with USP food grade 190 proof (95% ethanol, 5% water) ethanol being more preferred. Alcohol water contents in excess of 10% are less preferred, as then the additional water should be considered in relation to the total water content of the microemulsion 336 to prevent dissociation of the oil-soluble species from the modified oil phase droplets as discussed further below.

The alcohol may be present in the microemulsion 336 from 5% to 25% on a weight basis. Preferably, the alcohol constitutes from 10% to 23% of the microemulsion 336 on a weight basis. When the oil-soluble species is cannabis extracts, the alcohol constitutes from 16% to 22% of the microemulsion 336 on a weight basis.

The modified oil phase droplets of the microemulsion 336 may be considered to have a high alcohol content, thus having an oil to alcohol weight ratio of from 1:1.5 to 1:4, preferably from 1:1.5 to 1:3 by weight.

The modified polar continuous phase 322 includes a sugar or sugar alcohol and water. By “sugar or sugar alcohol” it is meant a sugar or a sugar alcohol preferably including from 3 to 12 carbon atoms that is a liquid at room temperature or soluble in water at room temperature. Preferable sugars include sucrose, cane sugar, and pure maple syrup, with pure maple syrup being preferred due to the inclusion of tree resins. Preferable sugar alcohols have from 3 to 6 carbon atoms and include glycerol (glycerin).

While one could expect additional sugar alcohols, including xylitol, erythritol, mannitol, and sorbitol to be useful in forming the microemulsion 336, all sugar alcohols are unexpectedly not interchangeable in forming shelf-stable and visually clear MOIW microemulsions, as xylitol, erythritol, mannitol, and sorbitol are not useful when both shelf-stable and visually clear microemulsions are desired. Thus, preferred sugar or sugar alcohols include sucrose, cane sugar, pure maple syrup, glycerol, and combinations thereof. More preferred sugar or sugar alcohols include pure maple syrup, glycerol, and combinations thereof. Presently, the most preferred sugar or sugar alcohol is glycerol.

When the sugar or sugar alcohol is glycerol, the ratio of glycerol to water is from 2:1 to 4.5:1 by weight, preferably from 3:1 to 4.5:1 by weight. When the sugar or sugar alcohol is pure maple syrup, sucrose, or cane sugar, and water is present in the syrup or used to solubilize the sucrose or cane sugar, this additional water becomes part of the water constituent of the microemulsion 336 and is thus included in the sugar or sugar alcohol to water weight ratio as water.

When the sugar or sugar alcohol is glycerol and the total water content of the microemulsion 336 is >10% to 25%, the glycerol may be present in the microemulsion 336 from 30% to 55%, preferably from 30% to 50% on a weight basis. When the total water content of the microemulsion 336 is >10% to 20%, the glycerol may be present in the microemulsion 336 from 30% to 50%, preferably from 30% to 45% on a weight basis. When the oil-soluble species is cannabis extracts, the glycerol constitutes from 35% to 45% of the microemulsion 336 on a weight basis.

The water of the polar continuous phase 332 is present in the microemulsion 336 from >10% to 25% on a weight basis. Preferably, water is present from >10% to 20% on a weight basis in the microemulsions 336. More preferably, water may be present in the microemulsion 336 from 11% to 16% on a weight basis. When the oil-soluble species is cannabis extracts, water is present in the microemulsion 336 from 12% to 15% on a weight basis. Water contents of 10% and less in the microemulsion 336 on a weight basis may result in dissociation of the oil-soluble species from the droplets, and thus a non-shelf-stable MOIW microemulsion.

The microemulsion 336 may optionally include other ingredients or “adjuvants” that are chemically compatible with the oil-soluble species and do not substantially interfere with the separation between the modified oil and water phases of the microemulsion. Such adjuvants may include hydrophilic or lipophilic gelling agents, thickeners, preservatives, antioxidants, electrolytes, perfumes, fillers, and pigments. Other adjuvants may be used in the microemulsion.

The following examples are provided to illustrate one or more preferred embodiments of the invention. Numerous variations can be made to the following examples that lie within the scope of the invention.

### EXAMPLES

### Example 1: Constituents of a MOIW Microemulsion Including Cannabis Extracts as the Oil-Soluble Species

A MOIW microemulsion was prepared having a 1 mL total volume. The MOIW microemulsion included approximately 10 mg of cannabis extracts including approximately 80% by weight CBD. The MOIW microemulsion also included from 30 mg to 100 mg of PC, from 150 mg to 250 mg of ethanol, from 300 mg to 550 mg of glycerin, and from 50 mg to 150 mg of medium chain triglycerides. TPGS was included to provide the desired physical structures in the MOIW microemulsion. In addition to these ingredients, the MOIW microemulsion included enough water to provide a total emulsion volume of 1 mL.

### Example 2: A Method of Making a MOIW Microemulsion Including Cannabis Extracts

Approximately 10 mg of CBD from cannabis extracts including approximately 80% by weight CBD was combined in MCT oil and then combined with TPGS, PC, glycerin, and ethanol in water. The combination was then mixed to form a MOIW microemulsion including the cannabis extracts.

### Example 3: Comparative Blood Uptake Rates for Intra-Oral Delivery of the Cannabis Extract CBD

Three intra-oral cannabis extract carrier systems were compared from a CBD blood uptake rate perspective. While CBD was used in this example as the oil-soluble species due to ready availability, the blood uptake rate data is believed to be similar for other oil-soluble species, such as THC, other cannabinoids, and terpenes. The first carrier system was a MOIW microemulsion consistent with Examples 1 and 2. The second carrier system was a conventional oil blend. The third carrier system was a conventional OIW emulsion.

The oil blend second carrier system was prepared by combining approximately 1.5% by weight cannabis extract including approximately 80% by weight CBD with approximately 98.5% by weight hemp seed oil to provide the conventional oil-only product. While hemp seed oil was used for this example, other oils including sunflower, olive, and MCT are expected to provide similar blood uptake rates.

The conventional OIW emulsion third carrier system included cannabis extract from the label claiming to include approximately 17 mg/mL CBD with the emulsion constituents being water, glycerin, MCT oil, natural lipids, the polysaccharides Xanthan and Acacia gum, Stevia, and potassium sorbate. The percentages of the OIW emulsion constituents were not known as the OIW emulsion was commercially obtained; however, the OIW emulsion was known to be a conventional, non-visually clear OIW emulsion. Independent testing revealed that the commercially obtained OIW emulsion in fact included 4.8 mg/mL CBD as opposed to the claimed approximately 17 mg/mL.

On an empty stomach, human subjects placed 1.2 mL of the MOIW microemulsion, 1 mL of the conventional oil blend, or 2.14 mL of the OIW emulsion including the cannabis extract under the tongue. Slightly different volumes were used so all dosages included approximately 10 mg of CBD. For the commercially obtained OIW emulsion, the 2.14 mL was used to provide the desired 10 mg CBD dose in view of the actual 4.8 mg/mL concentration of the emulsion. The subject held the liquid under the tongue for approximately 30 seconds to 2 minutes before swallowing.

Blood samples were collected from the subjects before the carrier system liquid was administered and at varying time intervals between approximately 20 and 180 minutes after intra-oral administration of the carrier system liquid. The collected blood samples were analyzed for the concentration of CBD using LCMS.

FIG. 4 provides the results of a CBD blood uptake rate and concentration analysis in graphical form. The time after administration of the carrier system liquid to the subjects when the blood sample was collected is represented on the X-axis, while the average nanograms (ng) of CBD per milliliter (mL) determined for the blood samples is represented on the Y-axis.

The MOIW Microemulsion line represents the blood uptake concentrations obtained with a MOIW microemulsion in accord with Examples 1 and 2. The oil blend line represents the blood concentrations obtained for the oil blend. The OIW Emulsion line represents the blood concentrations obtained for the conventional OIW emulsion.

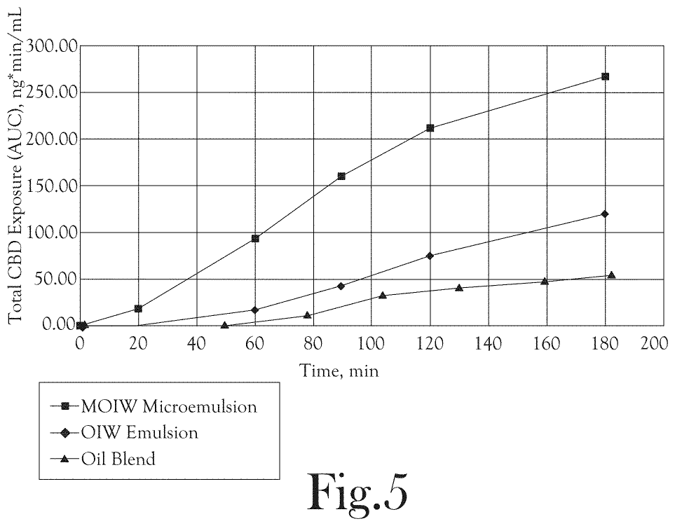

FIG. 5 provides the cumulative Area Under the Curve (AUC) values in ug*(min/mL) determined from a bloodstream concentration analysis in graphical form. The AUC values provide a measure of the cumulative amount of CBD in the bloodstream, thus total exposure across a period of time.

The superiority of the MOIW microemulsion at rapidly delivering CBD to the bloodstream in relation to the oil blend is readily apparent from a blood uptake perspective. Total CBD exposure over time was determined at different times after intra-oral introduction by multiplying the AUC up to the selected time point by the amount of CBD in the selected cannabis extract carrier system and dividing by a control value likewise calculated, thus (AUC1*carrier system1)/(AUCcontrol*carrier system control) at the selected time—thus calculating AUC as a cumulative amount of CBD in the bloodstream. As this calculation divides the total amount of CBD delivered by the numerator carrier system liquid by the total amount of CBD delivered by the denominator carrier system liquid, the resulting values relate to how many times more CBD was delivered by the numerator carrier system liquid in relation to the denominator carrier system.

The results from the AUC calculations are provided below in Table 1 for the MOIW microemulsion as the numerator and the oil blend as the denominator with estimation due to the slight variability in the withdrawal time of the blood samples.

TABLE 1

| Time | MOIW/Oil-Only |
|---|---|
| 0 | N/A |
| 20 | — |
| 60 | 22 |
| 90 | 10 |
| 120 | 7 |
| 180 | 5 |

At the 20-minutes after intra-oral introduction, a valid delivery comparison value could not be determined as the CBD measured in the blood resulting from the oil blend introduction was below the sensitivity of the LCMS instrument. As the MOIW microemulsion achieved an approximately 1.2 ng/mL blood concentration after introduction, and the oil blend achieved an essentially 0 blood concentration, the ability of the MOIW microemulsion to provide a significantly enhanced blood uptake rate in relation to the oil blend was established at the 20-minute time. Thus, with intra-oral introduction of a 1 mL dose of the MOIW microemulsion, the MOIW microemulsion can provide within 20-minutes a human subject blood CBD concentration from 0.3 to 1.5 ng/mL, preferably from 0.6 to 1.5 ng/mL, and more preferably from 0.8 to 1.5 ng/mL.

The significance of the MOIW microemulsion's 20-minute rapid delivery is seen at the 60-minute time point where the MOIW microemulsion had cumulatively delivered approximately 22 times more CBD to the bloodstream than the oil blend (peak concentrations of 2.9 ng/mL for the MOIW microemulsion vs. approximately 0.16 ng/mL for the oil blend). In fact, it was not until the 90-minute time point that the rate of increasing cumulative delivery provided by the MOIW microemulsion in relation to the oil blend was observed to slow. By cumulatively provided it is meant that the total bloodstream available CBD from introduction of the liquid carrier system to the human subject until the selected time. It was not until the approximately 180-minute mark that the oil blend is delivering CBD to the bloodstream at a rate comparable to that of the MOIW microemulsion.

As for the oil blend, the superiority of the MOIW microemulsion at rapidly delivering CBD to the bloodstream in relation to the conventional OIW emulsion also is apparent from a blood uptake perspective. The results from the AUC calculations are provided below in Table 2 for the MOIW microemulsion as the numerator and the OIW emulsion as the denominator with estimation due to the slight variability in the withdrawal time of the blood samples.

TABLE 2

| | MOIW/OIW |
|---|---|
| Time | Emulsion |
| 0 | N/A |
| 20 | — |
| 60 | 6.7 |
| 90 | 3.6 |
| 120 | 2.7 |
| 180 | 2.2 |

At the 20-minutes after intra-oral introduction, a valid delivery comparison value could not be determined as the CBD measured in the blood resulting from the OIW emulsion introduction was below the sensitivity of the LCMS instrument. As the MOIW microemulsion achieved an approximately 1.2 ng/mL blood concentration after introduction, and the OIW emulsion achieved an essentially 0 blood concentration, the ability of the MOIW microemulsion to provide a significantly enhanced blood uptake rate in relation to the OIW emulsion was established at the 20-minute time.

The significance of the MOIW microemulsion's 20-minute rapid delivery is readily seen at the 60-minute time point where the MOIW microemulsion had cumulatively delivered almost 7 times more CBD to the bloodstream than the OIW emulsion (peak concentrations of 2.9 ng/mL for the MOIW microemulsion vs. approximately 0.43 ng/mL for the oil blend). As with the oil blend, it was not until the approximately 180-minute mark that the OIW emulsion is delivering CBD to the bloodstream at a rate comparable to that of the MOIW microemulsion.

Of interest is that between the 60- and 90-minute time points the rate of increasing cumulative delivery decreased for the MOIW microemulsion while continuing to approximately linearly increase for the OIW emulsion. This is believed to illustrate the difference between the MOIW microemulsion's enhanced ability to intra-orally deliver to the bloodstream verses the OIW emulsion's favored gastrointestinal bloodstream delivery pathway. The substantially more rapid blood uptake provided by the MOIW microemulsion in relation to the OIW emulsion (20-minute time) shows the MOIW microemulsion's ability to substantially “bypass” the gut. Both the oil blend and the OIW emulsion are believed to substantially deliver the oil-soluble species via the gut while the MOIW microemulsion is believed to substantially deliver the oil-soluble species via the mouth and esophagus.

### Example 4: Total Exposure for Intra-Oral Delivery of the Cannabis Extract CBD

The three intra-oral cannabis extract carrier systems from Example 3 also were compared from a CBD total exposure perspective. With each extract carrier system approximately 10 mg of CBD was introduced to the subject. Thus, by comparing the AUC values for each of the three extract carrier systems within a selected timeframe, the total amount of cannabis extract to which the subject was exposed may be compared.

At the 60-minute time, the AUC was 94 ng*min/mL for the MOIW microemulsion, 14 for the conventional OIW emulsion, and 4.2 for the oil blend. Thus, at the 60-minute time point, of the approximately 10 mg of CBD consumed by the subject, the MOIW microemulsion had exposed the subject to nearly 7 times as much CBD as had the conventional OIW emulsion and approximately 22 times as much CBD as had the oil blend. The MOIW microemulsion was able to deliver to a human subject from 5 to 9 times, preferably from 6 to 8 times, more CBD to the bloodstream after 60-minutes than the OIW emulsion. The MOIW microemulsion was able to deliver to a human subject from 18 to 24 times, preferably from 20 to 22 times, more CBD to the bloodstream after 60-minutes than the oil blend.

At the 180-minute time, the AUC was 273 ng*min/mL for the MOIW microemulsion, 122 for the conventional OIW emulsion, and 58 for the oil blend. At the 180-minute mark the MOIW microemulsion and OIW emulsion carrier systems are delivering CBD to the bloodstream at similar rates, however from a cumulative and thus total delivery perspective, the MOIW microemulsion delivered more than twice as much CBD to the bloodstream as the OIW emulsion. At the 180-minute mark the oil blend carrier system is delivering CBD to the bloodstream at a somewhat faster rate than the MOIW microemulsion, however from a cumulative and thus total delivery perspective, the MOIW microemulsion delivered nearly 5 times as much CBD to the bloodstream as the oil blend.

At the 180-minute time, of the approximately 10 mg of CBD consumed by the subject, the MOIW microemulsion had exposed the subject to more than twice as much CBD as had the conventional OIW emulsion and almost 5 times as much CBD as had the oil blend. Thus, over the 180-minute timeframe, the MOIW microemulsion delivered at least 80%, preferably at least 100%, more CBD to the bloodstream as did the OIW emulsion and at least 4 times, preferably at least 5 times, as much CBD to the bloodstream as did the oil blend.

The data shows that for a subject to obtain similar cannabis extract exposure over a 3 hour timeframe to that provided by approximately 1 mL of the MOIW microemulsion, the subject would have to consume nearly 5 mL of the conventional OIW emulsion, or nearly 5 mL of the oil blend. Even in these “large dose” circumstances, the conventional OIW emulsion and the oil blend would be unlikely to provide the same 20-minute rapid onset bloodstream concentrations provided by the MOIW microemulsion. Thus, the ability of the MOIW microemulsion to efficiently deliver the cannabis extract to the bloodstream at substantially smaller dosing volumes was established.

### Example 5: Blood Uptake Consistency for Intra-Oral Delivery of the Cannabis Extract CBD

The MOIW microemulsion and oil blend cannabis extract carrier systems from Example 3 were compared from a CBD bloodstream uptake consistency perspective for 10 human subjects. Blood samples were collected from the subjects at varying time intervals between approximately 20 and 180 minutes after intra-oral administration of the carrier system liquid. The collected blood samples were analyzed for the concentration of CBD using LCMS. The standard deviation between the CBD blood concentrations was determined at each time point for the collected samples. The calculated standard deviation was then divided by the average CBD blood concentration determined at each time point to provide the percent standard deviation of the CBD blood concentrations. The determined values are provided below in Table 3.

TABLE 3

| Approximate Time | MOIW | Oil- |
|---|---|---|
| Minutes | Microemulsion | Only |
| 50-60 | 68% | 242% |
| 77-90 | 67% | 86% |
| 120-135 | 49% | 126% |
| 155-180 | 77% | 86% |
| AVG | 65% | 135% |

When the percent standard deviations are averaged for the MOIW microemulsion, an average percent standard deviation of 65% was determined. When the percent standard deviations are averaged for the oil blend, an average percent standard deviation of 135% was determined. Thus, the MOIW microemulsion provided less than half the variability in blood uptake in relation to the blood uptake provided by the oil blend. This substantial increase for the dosing reproducibility provided by the MOIW microemulsion in relation to the oil blend is believed attributable to the MOIW microemulsion's ability to provide relatively consistent intra-oral delivery in relation to the oil blend providing inconsistent blood uptake from reliance on GI adsorption.

We believe that the low and inconsistent uptake provided by conventional delivery systems coupled with the varying absorption ability of different subjects are factors in the inconsistent efficacy reports obtained from subjects using conventional oral cannabinoid delivery systems.

To provide a clear and more consistent understanding of the specification and claims of this application, the following definitions are provided.

Intra-oral delivery means that a substantial portion of the delivery into the bloodstream that occurs upon oral administration of the liquid including the deliverable occurs by transmucosal absorption through the mouth, throat, and esophagus before the liquid reaches the stomach. For droplets to be considered suitable for intra-oral delivery, the average droplet diameter is at most 125 nm. Intra-oral delivery is believed to increase with decreasing average droplet diameter, with average droplet diameters of approximately 50 nm being preferred.

An oil-soluble species is a species that is insoluble in water and soluble in medium chain triglyceride (MCT) oils at 50 mg/mL and higher, preferably 100 mg/mL and higher. Oil-soluble species are generally soluble in MCT oils at room temperature and are freely or very soluble in MCT oils at temperatures of 70 degrees Celsius and greater. The term “generally soluble in MCT oils at room temperature” is used because some high purity oil-soluble species are sparingly soluble in MCT oils at room temperature, but are freely or very soluble in the MCT oils above 70 degrees Celsius, and once solubilized in the MCT oils at elevated temperature, will remain solubilized at room temperature. Oil-soluble species are preferably pharmacologically active, more preferably are a drug or a supplement, and neither include nor are water. Thus, liquids and solids may exist that technically are soluble in oil, but because they also are soluble in water or not sufficiently soluble in MCT oils are not “oil-soluble species”.

Phosphatidylcholine (PC) molecules are a subset of the larger set of phospholipids and are commonly used to form liposomes in water. When placed in water without other constituents, PC forms liposomes. In the presence of an oil, the application of sufficient shear forces to the PC liposomes in water can produce monolayer structures, including micelles. PC has a head that is water-soluble and a tail that is much less water-soluble in relation to the head. PC is a neutral lipid, but carries an electric dipole moment of about 10 D between the head and the tail, making the molecule itself polar.

Tocopheryl polyethylene glycol succinate 1000 (TPGS) is generally considered a surfactant having a non-polar, oil-soluble “Vitamin E” tail and a polar, water-soluble polyethylene glycol head. TPGS is a member of the polyethylene glycol derivatives that also include polysorbate 20, 40, 60, and 80.

Room temperature and pressure means from 20 to 28 degrees Celsius at approximately 100 kPa.

Solid means a substance that is not a liquid or a gas at room temperature and pressure. A solid substance may have one of a variety of forms, including a monolithic solid, a powder, a gel, or a paste.

Liquid means a substance that is not a solid or a gas at room temperature and pressure. A liquid is an incompressible substance that flows to take on the shape of its container.

Solutions lack an identifiable interface between the solubilized molecules and the solvent. In solutions, the solubilized molecules are in direct contact with the solvent.

Solubilized means that the oil-soluble species to be delivered is in the solution of the droplet. When solubilized, dissociation (thus, liquid separation or solid formation) of the oil-soluble species does not result in droplet average particle diameters in excess of 200 nm as determined by DLS and discussed further below, or by the formation of precipitated crystals of the oil-soluble species visible with the naked eye. Thus, if either average particle diameters in excess of 200 nm or precipitated crystals visible to the naked eye form, the oil-soluble species is not solubilized in the solution of the droplet. If an oil-soluble species is not solubilized in the solution, it is insoluble in the solution. In many respects, solubility may be thought of as a concentration dependent continuum. For example, the following descriptive terms may be used to express solubility of a solute in a solvent (grams solid/mL of solvent) at 25 degrees Celsius:

| Descriptive Level | Parts solvent per 1 part of solute |
|---|---|
| Very Soluble | Less than 1 |
| Freely Soluble | From 1 to 10 |
| Soluble | From 10 to 30 |
| Sparingly Soluble | From 30 to 100 |
| Slightly Soluble | From 100 to 1000 |
| Very Slightly Soluble | From 1000 to 10,000 |
| Insoluble | More than 10,000 |

Dissociation occurs when a previously solubilized solid or liquid leaves a solution and is no longer in direct contact with a solvent of the solution. Dissociation of solids from the solvent occurs through recrystallization, precipitation, and the like. Dissociation of liquids from the solvent occurs through separation and the formation of a visible meniscus between the solvent and the dissociated liquid.

A shelf-stable microemulsion may be determined in one of two ways. One way to establish that a microemulsion stored in a sealed container substantially excluding air and moisture is shelf-stable is when dissociation of a solid does not occur and the oil phase droplets in the water do not change in average diameter by more than +/−20% at about 25° C. for a time period of at least 3 months to 2 years, preferably for a time period of at least 6 months to 2 years, and more preferably, for a time period of at least 1 year to 2 years. Another way to establish that a microemulsion is shelf-stable is when dissociation of a solid does not occur and the oil phase droplets in the water do not separate into a visibly distinct phase with a visible meniscus when stored in a sealed container substantially excluding air and moisture at about 25° C. for a time period of at least 6 months to 2 years, and more preferably, for a time period of at least 1 year to 2 years. Either type of dissociation means that the microemulsion is not shelf-stable.

A visually clear microemulsion has an average particle diameter of 200 nm and less and lacks precipitated solid crystals visible to the naked eye.

Emulsions are mixtures of two or more liquids that do not solubilize. Thus, one of the liquids carries droplets of the second liquid. The droplets of the second liquid may be said to be dispersed in a continuous phase of the first liquid. An interface, separation, or boundary layer exists between the carrier liquid (continuous phase) and the droplets of the second liquid. Emulsions may be macroemulsions, pseudo-emulsions, microemulsions, or nanoemulsions. The primary differences between macroemulsions, microemulsions, and nanoemulsions are the average diameter of the droplets dispersed in the continuous phase and the stability of the emulsion over time. Pseudo-emulsions are differentiated as solids are present in the emulsion.

Droplets or liquid particles are formed by the hydrophobic “oil” phase of a microemulsion and are carried by the hydrophilic continuous phase. The exterior of the droplets is defined by a boundary layer that surrounds the volume of each liquid droplet. The boundary layer of a droplet defines the exterior surface of the droplets forming the dispersed oil phase of the microemulsion. The continuous phase of the microemulsion resides exterior to the boundary layer of the droplets, and thus, carries the droplets.

Macroemulsions are thermodynamically unstable but kinetically stable dispersions of oil in water, with oil being defined as any water-insoluble liquid. By thermodynamically unstable it is meant that once created, the macroemulsion is always reverting to the original, immiscible state of the oil and water constituents (demulsification), but this break down is slow enough (thus, kinetically “stable”) that the macroemulsion may be considered stable from an intended use practicality perspective. Macroemulsions scatter light effectively and therefore appear milky, because their droplets are greater in diameter than the wavelength of visible light. The droplets of a macroemulsion usually have average droplet diameters from 10 to 50 micrometers. The IUPAC definition of a macroemulsion is an “emulsion in which the particles of the dispersed phase have diameters from approximately 1 to 100 micrometers. Macro-emulsions comprise large droplets and thus are “unstable” in the sense that the droplets sediment or float, depending on the densities of the dispersed phase and dispersion medium.”

Pseudo-emulsions are dispersions of oil in water, with oil being defined as any water-insoluble liquid, including tiny (micronized) solid granules that are not fully solubilized in the oil droplets. The term “pseudo-emulsion” is used as these mixtures are not true emulsions as the solid granules are not fully solubilized into the droplets. The droplets of a pseudo-emulsion have an average droplet diameter of 1 to 20 micrometers, thus being a “solid granule modified macroemulsion”.

Microemulsions are thermodynamically stable dispersions of oil in water, with oil being defined as any water-insoluble liquid. Microemulsion are made by simple mixing of the components. Thus, microemulsions spontaneously form and do not require high shear forces. Unlike macroemulsions, microemulsions do not substantially scatter light. The IUPAC definition of a microemulsion is a “dispersion made of water, oil, and surfactant(s) that is an isotropic and thermodynamically stable system with dispersed domain diameter varying approximately from 1 to 100 nm, usually 10 to 50 nm.” Thus, the droplets of a microemulsion are approximately three orders of magnitude smaller than the droplets of a macroemulsion and are thermodynamically stable.

Nanoemulsions have average droplet diameters from 10 to 125 nanometers, thus being at least an order of magnitude smaller in average droplet diameters than macro- and pseudo-emulsions. Transparent nanoemulsions have average droplet diameters from 10 to 100 nanometers. Nanoemulsions are made with mechanical, high shear forces. While the average droplet diameter of nanoemulsions and microemulsions formally overlap, in practice, the average droplet diameter of nanoemulsions are or become larger than those of microemulsions, as lacking the thermodynamic stability of microemulsions, the average droplet diameter of nanoemulsions is forever increasing.

Continuous phase means the portion of a microemulsion that carries the droplets that include the substance to be delivered. For example, the modified oil-in-water microemulsions (non-polar droplets in polar continuous phase) addressed herein have oil droplets including the oil-soluble species to be delivered carried in a polar, “water” continuous phase. While the words “water” and “oil” are used, the “water” can be any liquid that is more polar than the “oil” (such as a polar oil), and the “oil” can be any liquid that is less polar than the “water. Thus, the terms “polar continuous phase” and “water continuous phase” are synonymous, unless water is specifically being discussed as one of the microemulsion components.

Average droplet diameter is determined by dynamic light scattering, sometimes referred to a photon correlation spectroscopy. The determination is made between 20 and 25 degrees Celsius. One example of an instrument suitable for average droplet diameter determination is a Nicomp 380 ZLS particle sizer as available from Particle Sizing Systems, Port Richey, Fla. DLS can determine the diameter of droplets in a liquid by measuring the intensity of light scattered from the droplets to a detector over time. As the droplets move due to Brownian motion the light scattered from two or more droplets constructively or destructively interferes at the detector. By calculating the autocorrelation function of the light intensity and assuming a droplet distribution, it is possible to determine the sizes of droplets from 1 nm to 5 um. The instrument is also capable of measuring the Zeta potential of droplets.

Ingestible means capable of being ingested through the mouth by a living mammal while edible means fit to be eaten, thus in contrast to being unpalatable or poisonous. Edible also means that the composition has less than the permitted amount of viable aerobic microorganisms and meets the American Herbal Products Association (AHPA) guidelines for metals, adulterants, toxins, residual solvents, and pesticides.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

While various aspects of the invention are described, it will be apparent to those of ordinary skill in the art that other aspects and implementations are possible within the scope of the invention. Accordingly, the invention is not to be restricted except in light of the attached claims and their equivalents.

1. A composition comprising:
an oil-soluble species; and
a modified oil-in-water microemulsion including a modified oil phase and a modified polar continuous phase,
where the oil-soluble species is solubilized in the modified oil phase, the modified oil phase comprising a phospholipid, a polyethylene glycol derivative, an oil, and an alcohol, and
where the modified polar continuous phase comprises a sugar or sugar alcohol and water.

2. The composition of claim 1, where the modified oil-in-water microemulsion is visually clear.

3. The composition of claim 1, where the modified oil-in-water microemulsion is shelf-stable.

4. The composition of claim 1, where the modified oil-in-water microemulsion is ingestible and edible.

5. The composition of claim 1, the modified oil-in-water microemulsion configured to provide uptake of the oil-soluble species to the bloodstream of a mammal at an effective bloodstream concentration through the oral and gastric mucosa of the mammal.

6. The composition of claim 1, where the modified oil phase is configured to better solubilize the oil-soluble species than the oil alone.

7. The composition of claim 1, where the modified oil phase is dispersed in the modified polar continuous phase.

8. The composition of claim 7, where droplets of the modified oil phase have an average droplet diameter of 1 to 100 nanometers.

9. The composition of claim 7, where droplets of the modified oil phase have an average droplet diameter of 7 to 30 nanometers.

10. The composition of claim 1, where the oil-soluble species is chosen from cannabis extracts, terpenes, and combinations thereof.

11. The composition of claim 10, the cannabis extracts chosen from cannabidiol, tetrahydrocannabinol, other cannabinoids, and combinations thereof.

12. The composition of claim 10, where the cannabis extracts comprise cannabidiol and tetrahydrocannabinol.

13. The composition of claim 10, the cannabis extracts chosen from cannabidiol, tetrahydrocannabinol, and combinations thereof.

14. The composition of claim 10, the terpenes chosen from limonene, pinene, linalool, beta-caryophyllene, retinol, phytol, myrcene, humulene, ocimene, terpinolene, geraniol, geranylgeraniol, and combinations thereof.

15. The composition of claim 10, where the terpenes comprise beta-caryophyllene.

16. The composition of claim 1, further comprising an alcohol-soluble deliverable in the modified oil phase, the alcohol-soluble deliverable chosen from a plant sterol, a polyphenol, an anti-microbial, and combinations thereof.

17. The composition of claim 16, the plant sterol chosen from tribulus terrestris, yohimbe, and combinations thereof.

18. The composition of claim 16, the polyphenol chosen from resveratrol, pterostilbene, curcumin, Boswellia, quercetin, and combinations thereof.

19. The composition of claim 16, the anti-microbial chosen from artemisinin, monolaurin, Andrographis, and combinations thereof.

20. The composition of claim 1, where the phospholipid is a glycerophospholipid isolated from lecithin.

21. The composition of claim 20, where the phospholipid is chosen from phosphatidylcholine, phosphatidylethanolamine, phosphatidylinositol, ceramide phosphoryl ethanolamine, ceramide phosphoryl choline (SPH), and combinations thereof.

22. The composition of claim 20, where the phospholipid is chosen from phosphatidylcholine, phosphatidylethanolamine, and combinations thereof.

23. The composition of claim 20, where the phospholipid is at least 80% by weight phosphatidylcholine.

24. The composition of claim 1, where the polyethylene glycol derivative is chosen from polyethylene glycol modified vitamin E, polysorbate 40, polysorbate 60, polysorbate 80, and combinations thereof.

25. The composition of claim 24, where the polyethylene glycol modified vitamin E is tocopheryl polyethylene glycol succinate 1000.

26. The composition of claim 24, where the polyethylene glycol derivative is chosen from tocopheryl polyethylene glycol succinate 1000, polysorbate 60, polysorbate 80, and combinations thereof.

27. The composition of claim 1, where the polyethylene glycol derivative is tocopheryl polyethylene glycol succinate 1000.

28. The composition of claim 1, the oil chosen from a medium chain triglyceride, a citrus oil, and combinations thereof.

29. The composition of claim 28, the medium chain triglyceride chosen from caproic acid (hexanoic acid), caprylic acid (octanoic acid), capric acid (decanoic acid), lauric acid (dodecanoic acid), and combinations thereof.

30. The composition of claim 28, the medium chain triglyceride chosen from caprylic acid, capric acid, and combinations thereof.

31. The composition of claim 28, the citrus oil chosen from orange oil, lemon oil, and combinations thereof.

32. The composition of claim 1, where the alcohol is 95% ethanol by weight.

33. The composition of claim 1, the sugar or sugar alcohol chosen from sucrose, cane sugar, pure maple syrup, glycerol, and combinations thereof.

34. The composition of claim 1, the sugar or sugar alcohol chosen from pure maple syrup, glycerol, and combinations thereof.

35. The composition of claim 1, where the sugar or sugar alcohol is glycerol.

36. The composition of claim 1, where the oil-soluble species comprises from 1% to 4% of the composition by weight.

37. The composition of claim 1, where the oil-soluble species comprises from 1% to 3% of the composition by weight.

38. The composition of claim 1, where a ratio of the phospholipid, to the oil, to the polyethylene glycol derivative, to the alcohol, to the sugar or sugar alcohol, and to the water is 1:2:0.6-3.3:4:7-9:2-3.5±20% by weight.

39. The composition of claim 1, where a ratio of the phospholipid, to the oil, to the polyethylene glycol derivative, to the alcohol, to the sugar or sugar alcohol, and to the water is 1:2:0.6-3.3:4:7-9:2-3.5±10% by weight.

40. The composition of claim 1, the modified oil phase further comprising an oil, where a ratio of the oil to the oil-soluble species is 1:0.05 to 0.3±10% by weight.

41. The composition of claim 1, the modified oil phase further comprising an oil, where a ratio of the oil to the oil-soluble species is 1:0.05 to 0.3±5% by weight.

42. The composition of claim 1, where the phospholipid comprises from 2% to 10% of the composition by weight.

43. The composition of claim 1, where the polyethylene glycol derivative comprises from 5% to 15% of the composition by weight.

44. The composition of claim 1, where a ratio of the phospholipid to the polyethylene glycol derivative is 1:0.4 to 1:4 by weight.

45. The composition of claim 1, where a ratio of the phospholipid to the polyethylene glycol derivative is 1:1.6 to 1:4 by weight.

46. The composition of claim 1, where the oil comprises from 5% to 15% of the composition by weight.

47. The composition of claim 1, where the alcohol comprises from 5% to 25% of the composition by weight.

48. The composition of claim 1, where a ratio of the oil to the alcohol is 1:1.5 to 1:4 by weight.

49. The composition of claim 1, where the sugar or sugar alcohol comprises from 30% to 55% of the composition by weight.

50. The composition of claim 1, where the sugar or sugar alcohol comprises from 30% to 45% of the composition by weight.

51. The composition of claim 1, where the water comprises from >10% to 25% of the composition by weight.

52. The composition of claim 1, where the water comprises from 12% to 15% of the composition by weight.

53. The composition of claim 1, the composition configured to provide a human subject a from 0.3 to 1.5 ng/mL blood concentration of the oil-soluble species within 20-minutes of intra-orally introducing the composition including 10 mg of the oil-soluble species to the human subject.

54. The composition of claim 1, the composition configured to provide a human subject with a blood concentration from 0.3 ng/mL to 1.5 ng/mL of the soil-soluble species within 20-minutes of intra-oral administration of the composition to the human subject.

55. The composition of claim 1, the composition configured to provide a human subject with a blood concentration from 0.8 ng/mL to 1.5 ng/mL of the oil-soluble species within 20-minutes of intra-oral administration of the composition to the human subject.

56. The composition of claim 1, the composition configured to provide a human subject from 18 to 24 times more oil-soluble species in the bloodstream within 60-minutes of intra-oral administration than when the oil-soluble species is orally delivered to the human subject in an oil blend.

57. The composition of claim 1, the composition configured to provide a human subject at least 4 times as much oil-soluble species to the bloodstream within 180-minutes of intra-oral administration than when the oil-soluble species is orally delivered to the human subject in an oil blend.

58. The composition of claim 1, the composition configured to provide a human subject from 5 to 9 times more oil-soluble species in the bloodstream within 60-minutes of intra-oral administration than when the oil-soluble species is orally delivered to the human subject in an OIW emulsion.

59. The composition of claim 1, the composition configured to provide a human subject at least twice as much oil-soluble species to the bloodstream within 180-minutes of intra-oral administration than when the oil-soluble species is orally delivered to the human subject in an OIW emulsion.

60. A method of making a modified oil-in-water microemulsion for intra-oral delivery of an oil-soluble species to the bloodstream of a human subject, the method comprising:
combining a phospholipid, a polyethylene glycol derivative, an oil, and an alcohol to form an alcohol-lipid mixture;
combining a sugar or sugar alcohol and water to form a modified polar continuous phase; and
combining the oil-soluble species with the alcohol-lipid mixture and the modified polar continuous phase at atmospheric pressure to form the modified oil-in-water microemulsion.

61.-66. (canceled)

67. A method of delivering an oil-soluble species to the bloodstream of a human subject, the method comprising:
introducing intra-orally to a human subject a composition comprising:
an oil-soluble species, and
a modified oil-in-water microemulsion including a modified oil phase and a modified polar continuous phase,
where the oil-soluble species is solubilized in the modified oil phase, the modified oil phase comprising a phospholipid, a polyethylene glycol derivative, an oil, and an alcohol, and
where the modified polar continuous phase comprises a sugar or sugar alcohol and water; and
delivering the oil-soluble species to the bloodstream of the human subject,
where within 20-minutes of the introducing the composition intra-orally to the human subject, approximately 1 mL the composition provides the human subject a blood concentration from 0.3 to 1.5 ng/mL of the oil-soluble species.

68. (canceled)
